(19) United States
(12) Patent Application Publication
(10) Pub. No.: US 2009/0035808 A1
(43) Pub. Date: Feb. 5, 2009

(54) MEDIUM FOR DETECTING MICROORGANISMS

(76) Inventors: Hans-Werner Zoch, Wachmannstrasse (DE); Thomas Koch, Isarstrasse (DE); Andre Walter, Zaunkonigweg (DE); Ekkard Brinksmeier, Parkallee (DE); Jan Kuver, Schwabisch-Hall-Strasse (DE); Andreas Rabenstein, Helmer (DE)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 12/064,183

(22) PCT Filed: Sep. 11, 2006
(86) PCT No.: PCT/EP2006/008821

§ 371 Date: Jul. 8, 2008

(30) Foreign Application Priority Data
Sep. 9, 2005 (EP) 05019670-8

(51) Int. Cl. C12Q 1/06; C12Q 1/04; C12N 1/00; C12M 1/00; C12M 1/22; C12M 1/24

(52) U.S. Cl. 435/39; 435/34; 435/243; 435/289.1; 435/304.1; 435/305.1

(57) ABSTRACT

Provided is a culture medium for microorganisms present in contaminated working fluids such as coolants. More particularly, said culture medium is particularly suitable for supporting growth of microorganisms colonizing metalworking fluids and allows for specific detection of both bacterial microorganisms and fungal microorganisms the latter depending on the added selective agents which can be antibiotics for the detection of fungal contamination or fungicides for the detection of bacterial contamination. Furthermore, devices and kits comprising the culture medium of the present invention are described as well as a method of detecting microbial contamination of metalworking fluids.

### FIELD OF THE INVENTION

The present invention relates to new means and methods for detecting microorganisms in possibly contaminated working fluids such as cooling lubricants and improved processes for the antimicrobial treatment of such fluids. In particular, the present invention relates to a culture medium for detecting microorganisms comprising at least one fatty component or derivative thereof. The present invention further concerns a device comprising the culture medium of the present invention, wherein said device is suitable for use in detection of microorganisms in a possibly contaminated material. Furthermore, the present invention relates to a kit comprising the culture medium of the invention. The present invention further concerns a method of detecting microorganisms in a possibly contaminated material. Furthermore, the present invention relates to the use of the above-mentioned culture medium, device and kit in a method of detecting microorganisms in a possibly contaminated material.

### BACKGROUND OF THE INVENTION

In metal-cutting manufacturing the use of water miscible metalworking fluids is not only common practice but also indispensable primarily preventing an overheating of the workpiece and the tool(s) due to friction during manufacturing processes. Metalworking fluids (MWFs) are oils or water-based fluids. They are sometimes referred to as suds, coolants, cooling lubricants, slurry or soap. Metalworking fluids are used during the machining and shaping of metals and provide for cooling of the workpiece and the tool(s), lubricating the contact of the cutting edge during evacuation of the splinters and, if applicable, applying of appropriate power additives. Certain metalworking fluids feature inverse solubility, wherein the material becomes less soluble in water as the temperature of the solution increases: when a respective metalworking fluid comes in contact with the hot workpiece and tool(s), certain components of the metal working fluid come out of solution and coat the metal surfaces with a concentrated film, e.g. a lubricant film. Therefore, metalworking fluids may be able to provide both lubricity and heat removal.

Metalworking fluids are always exposed to microbial attack and comprise organic matter which underlies degradation and metabolic processes mediated by said microbial attacker. Even with properly conducted operations it is virtually impossible to prevent colonization with bacteria, yeasts or other fungi. Microbial colonization moreover takes place through the mixing water, the surfaces of tools and machines, the skin and clothing of the operatives or directly from the air. As a result, even if gradually, microorganisms impair the functioning of the metalworking fluid considerably. This loss of function is manifested, depending on the extent of the colonization, by odor formation, a fall in pH, a reduction in the corrosion prevention capacity, a change in the dispersity and thus in the filtration characteristics and instability of the emulsion. The metalworking fluid becomes unusable thereby.

In consideration of the sales figures of about 600.000 tons metalworking fluid emulsion per year, for example, in Germany the microbial damage of water-mixed metalworking fluids becomes a serious problem in the manufacturing technology, causing for example additional and increasing costs for the companies due to shut down times while changing the composition and cleaning the facilities, shortened operating life of the metalworking fluid, impairment of the work result and increasing amounts of wastage. Furthermore, metalworking fluids, if contaminated with microorganisms constitute a significant respiratory hazard, especially for exposed employees in the metal working industry.

Therefore, microbial contamination of metalworking fluids and associated machinery (such as washing machines) and pipework has to be monitored and controlled. Direct means of measuring bacterial contamination are consistently based on the use of conventional culture media which may be used in combination with investigation of other chemical, physical or physiological parameters indirectly reflecting the fluid's quality, e.g. fluid concentration or pH or content of ATP, which is an universal, ubiquitously present power supplier. For example, the significance of the pH as regards the degree of microbial damage in a metalworking fluid is based on the assumption that the acid-base-balance in the metalworking fluid will be affected significantly by the intermediate catabolic products occurring during microbial degradation of the metalworking fluid.

However, the culture media used so far to detect microbial account in metalworking fluids are initially derived from the field of medical and hygienic monitoring. Said commercially available culture media typically comprise microbiological standard media. Standard media provide a basis for a general detection method for microorganisms. As to their unspecific chemical composition and the high nutrient content the standard media act mostly very highly selective and thus may allow for an accelerated and preferred growth of specific germs resulting in adulterating the microbial profiling of the material to be tested. Therefore, the common, commercially available culture media are not suitable to detect organisms, being yielded from specialized environmental conditions such as metalworking fluids. Accordingly, there is a need to adapt the culture medium on the specialized condition predominating the metalworking fluids to provide for reliable, reproducible, standardized detection methods allowing the detection and identification of the whole entirety of the microorganisms colonizing metalworking fluids in order to enable the timely discharging of counter measures.

In view of the need of saving of costs and providing for maximum safety at workplaces in metal-cutting manufacturing, the technical problem of the present invention is to provide specialized means and methods for an accurate microbiological profiling of microbial affection in metalworking fluids which allow for identifying the origin of microbial contamination. The solution to said technical problem is achieved by providing the embodiments characterized in the claims, and described further below.

### SUMMARY OF THE INVENTION

The present invention is directed to a culture medium for microorganisms comprising at least one fatty component or derivative thereof. In particular, said fatty component or derivative thereof comprises one or more fatty acids or an emulsifying agent or derivatives thereof. Furthermore, the invention is directed to a device comprising the culture medium of the present invention or one or more components thereof. The present invention is also directed to a kit comprising the culture medium of the present invention or one or more components thereof, or the device of the present invention. Furthermore, the present invention relates to a method of detecting contamination of coolants with microorganisms, said method comprising the use of the culture medium or the device of the present invention. The present invention is further directed to the use of the culture medium or of one or more of its components, of the device or of the kit of the invention for any method of the present invention.

### DETAILED DESCRIPTION OF THE INVENTION

The present invention generally relates to a culture medium useful for the detection of microorganisms, i.e. bacteria and fungi in water miscible metalworking fluids.

The present invention is based on observations that a culture medium for microorganisms comprising at least one fatty component or derivative thereof, which can be usually found in water miscible metalworking fluids such as coolants, is capable to allow for the improved growth and subsequent detection of the whole entirety of the microorganisms colonizing metalworking fluids. Preliminary experiments suggest a surprising effect as to the formulation of the culture medium of the present invention which provides reliable, reproducible, standardized microbiological detection methods.

Thus, in a first embodiment the present invention relates to a culture medium for microorganisms comprising at least one fatty component or derivative thereof.

As used herein, the term “metalworking fluid” generally refers to a basic fluid in the form of liquid hydrocarbon compounds with various properties and tasks, which contain other substances according to their use. One prominent example are cooling lubricants or coolants. Accordingly, in DIN 51 385, cooling lubricants are defined and classified according to their use. In practice, it is usually sufficient to classify metalworking fluids into those which are miscible with water and those which are not miscible with water. Water-based metalworking fluids comprise both oil-in-water emulsions and pure solutions, wherein the water content ranges between 90 and 98%, depending on the respective operating conditions. The basic substances used for metalworking fluids are both mineral oils and oils from natural raw materials. Mineral oils consist predominantly of paraffin hydrocarbons, naphthenic hydrocarbons and aromatic hydrocarbons. Oils from natural raw materials contain, besides triglycerides, also concomitant substances such as, for example, free fatty acids, phosphates, protein, carbohydrates, waxes, coloring matter or aromatic-containing hydrocarbons. Most of these unwanted concomitant substances are removed by refining. Also removed thereby are natural inhibitors such as, for example, tocopherols. The metalworking fluids may also contain, to improve the use properties, additives such as adhesion promoters, emulsifiers, antifoams, additives for high-pressure lubrication, corrosion preventives, detergents and viscosity index improvers. However, other metalworking fluids may be assessed as well for example coolants, cooling lubricants, borehole flushing fluids, cutting fluids, rolling fluids, hydraulic fluids, heat transfer media and wood protection agents.

As used herein, the term “microorganism” generally relates to any microorganism whose presence or absence is detectable and intended to be detected in accordance with the present invention. In particular, the microorganisms to be detected can be bacteria, fungi, and/or yeast. In one preferred embodiment, the microorganisms to be detected are mycobacteria.

As used herein, the term “culture medium” is generally related to a nutrient source which allows microorganisms to grow, wherein said nutrient source is specifically adapted on the specialized conditions predominating the metalworking fluids allowing for a complete microbial profiling of the probably colonized metalworking fluids. Said nutrient source may provide for vitamins, amino acids, trace elements, salts, including all molecules, compounds and substances classified in each category by those skilled in the art whether organic or inorganic, and the categories are not intended to exclude any substance which may be necessary for or conductive to maintain life.

Without intended to be bound by theory, it is believed that the culture medium of the present invention allows for more exactly and more rapidly obtainable results by determining the microbial count in metalworking fluids.

The fact that each culture medium being capable of allowing microorganisms to grow may be selective is well known to the person skilled in the art in the technical field of microbiology. In the present invention a culture medium on the basis of water miscible metalworking fluids is implemented. The culture medium contains those ingredients of water miscible metalworking fluids, which proved to be well metabolizable by typical metalworking fluid colonizers. The benefit of the culture medium of the present invention lies in its formulation which provides for reliable and reproducible methods for detecting absence or presence of microorganisms in a probably contaminated metalworking fluid and allows the person in charge to take counter measures in time.

Thus, the present invention provides for a culture medium, wherein the fatty component or derivative thereof comprises one or more fatty acids or derivatives thereof.

In the context of the invention, particular significance is attributed to the term “fatty acid”, which may comprise any of a class of aliphatic monocarboxylic acids that form part of a lipid molecule and can be hydrolytically derived from fats, having the general formula CₙH₂ₙ₊₁COOH. The fatty acids according to the present invention preferably have an even number of carbon atoms. In accordance with the present invention fatty acids can be any of the organic carboxylic acids present in fats and oils as esters of glycerol, wherein the fatty acids can be saturated, i.e., each carbon atom is connected to its carbon atom neighbors by single bonds or unsaturated, i.e., contain at least one carbon-carbon double bond. The fatty acids of the present invention also comprise industrially manufactured fatty acids which can be produced, for example by the hydrolysis of the ester linkages in a fat or biological oil (both of which are triglycerides), with the removal of glycerol. In the case the fatty acid of the present invention is a saturated fatty acid, said fatty acid is characterized in that it may not contain any double bonds or other functional groups along the chain. The term “saturated” refers to hydrogen, in that all carbons (apart from the carboxylic acid [—COOH] group) contain as many hydrogens as possible. In other words, the omega (ω) end contains 3 hydrogens (CH₃—) and each carbon within the chain contains 2 hydrogens (—CH₂—). Examples for saturated fatty acids include, but are not limited to: butyric (CH₃(CH₂)₂COOH), lauric (dodecanoic acid, CH₃(CH₂)₁₀COOH), myristic (tetradecanoic acid, CH₃(CH₂)₁₂COOH), palmitic (hexadecanoic acid, CH₃(CH₂)₁₄COOH), stearic (octadecanoic acid, CH₃(CH₂)₁₆COOH), arachidic (eicosanoic acid, CH₃(CH₂)₁₈COOH). In the case the fatty acid of the present invention is a unsaturated fatty acid, said fatty acid is characterized in that one or more alkene functional groups exist along the chain, with each alkene substituting a singly-bonded —CH₂—CH₂— part of the chain with a doubly-bonded —CH═CH— portion (that can be a carbon double bonded to another carbon). In most of these, each double bond has 3n carbon atoms after it, for some n, and are all cis bonds. Examples of unsaturated fatty acids include but are not limited to: (alpha)-linolenic acid (CH₃CH₂CH═CHCH₂CH═CHCH₂CH═CH(CH₂)₇COOH), arachidonic acid (CH₃(CH₂)₄CH═CHCH₂CH═CHCH₂CH═CHCH₂CH═CH(CH₂)₃COOH), oleic acid (CH₃(CH₂)₇CH═CH(CH₂)₇COOH), erucic acid (CH₃(CH₂)₇CH═CH(CH₂)₁₁COOH), linoleic acid (CH₃(CH₂)₄CH═CHCH₂CH═CH(CH₂)₇COOH). Furthermore, the fatty acid of the present invention can be an essential fatty acid. Essential fatty acids are the polyunsaturated fatty acids, linoleic acid and alpha-linolenic acid, which are the parent compounds of the omega-6 and omega-3 fatty acid series, respectively. The fatty acid of the present invention can also be a trans fatty acid, commonly shortened to trans fat, which is an unsaturated fatty acid molecule that contains a trans double bond between carbon atoms, which makes the molecule less kinked compared to fatty acids with cis double bonds. The fatty acid of the present invention can also be a free fatty acid or uncombined fatty acid, which can be characterized in that it is not bound or attached to other molecules, like triglycerides or phospholipids. Such a free fatty acid may come from the breakdown of a triglyceride into its components (fatty acids and glycerol). The term “fatty acid” also comprises compounds which are not denoted as “fatty acids” in the common sense but which have identical or similar properties and which preferably are able to substitute any one of the above described fatty acids in context with the media used in the appended examples.

In a preferred embodiment of the present invention the fatty acids are selected from the group consisting of all even-numbered fatty acids C14 to C20, which are saturated, mono- or polyunsaturated.

In another preferred embodiment the present invention comprises a culture medium, wherein the fatty component or derivative thereof comprises an emulsifying agent or derivative thereof. As used herein the term “emulsifying agent”, commonly also referred to as a surfactant or surface active material, is a substance which stabilizes an emulsion. Emulsifying agents are generally well known and commercially available. An example of an naturally occurring emulsifying agent is lecithin. Emulsifying agents according to the present invention comprise ionic and non-ionic emulsifying agents, wherein the ionic emulsifying agents can be selected from anionic, cationic and amphoteric emulsifying agents, the latter comprising ampholytes as well as betaines. Emulsifying agents according to the present invention also comprise detergents which are another class of surfactant, and will bind to both oil and water, thus holding microscopic oil droplets in suspension. Emulsifying agents in accordance with the present invention may also comprise biocompatible surfactants.

Anionic surfactants, mixtures of anionic and nonionic surfactants and nonionic surfactants alone may be used in accordance with the present invention. Suitable anionic surfactants are readily biodegradable anionic surfactants, such as soaps for example. Alkylsulfates, more particularly fatty alcohol sulfates, may also be used. Anionic surfactants based on petrochemicals, such as alkylbenzenesulfonate or alkylethersulfates, for example, are less suitable. Preferred nonionic surfactants are alkyl glycoside compounds which have preferably been obtained from straight-chain fatty alcohols containing at least 8 carbon atoms. However, biosurfactants of biological origin may be used in addition to or instead of the surfactants mentioned above. Examples of biosurfactants are sophorose lipid, trehalose lipid or lipopeptides of the type known as metabolism products or membrane constituents of a number of microorganism strains. Sorbitan esters, for example sorbitan monostearate or sorbitan monooleate, may be used instead of or in addition to the nonionic surfactants mentioned above.

The surfactant compounds mentioned above are present in the nutrient concentrates according to the invention in quantities of, typically, 0.02-2.0% weight per volume (w/v), preferably 0.1 to 1.0%, more preferably 0.2 to 0.5% and most preferred quantities of up to about 0.25% w/v of surfactant are sufficient to allow for the metalworking fluid colonizers to grow.

Thus, in a particular preferred embodiment the emulsifying agent or derivative thereof comprises fatty alcohols or derivatives thereof. As used herein the term “fatty alcohols” relates to alcohols derived from natural fats and oils. Examples for fatty alcohols used in accordance with the present invention include but are not limited to erucyl alcohol, ricinolyl alcohol, arachidyl alcohol, capryl alcohol, capric alcohol, behenyl alcohol, lauryl alcohol (1-dodecanol), myristyl alcohol (1-tetradecanol), cetyl (or palmityl) alcohol (1-hexadecanol), stearyl alcohol (1-octadecanol), isostearyl alcohol, oleyl alcohol (cis-9-octadecen-1-ol), palmitoleyl alcohol, linoleyl alcohol (9Z,12Z-octadecadien-1-ol), polyunsaturated, elaidyl alcohol (9E-octadecen-1-ol), elaidolinoleyl alcohol (9E,12E-octadecadien-1-ol), linolenyl alcohol (9Z,12Z,15Z-octadecatrien-1-ol), elaidolinolenyl alcohol (9E,12E,15-E-octadecatrien-1-ol). It goes without saying that the term “fatty alcohol” also comprises compounds which are not denoted as “fatty alcohols” in the common sense but which have identical or similar properties and which preferably are able to substitute any one of the above described fatty alcohols in context with the media used in the appended examples.

In a more particular preferred embodiment the emulsifying agent or derivative thereof comprises glycerol or a derivative thereof.

In a most preferred embodiment the emulsifying agent or derivative thereof comprises fatty alcohol ethoxylate or derivatives thereof. As used herein, the term “fatty alcohol ethoxylate” refers to, for example, fatty alcohol ethoxylates of the formula RO(Etox)ₙ, wherein R can be a linear or non-linear Cₙ, alkyl chain, and n representing the weighted average ethoxylation degree.

In another most preferred embodiment the emulsifying agent or derivative thereof comprises a mixture of fatty alcohol ethoxylate, preferably of 2 ethylene oxide/5 ethylene oxide fatty alcohol ethoxylate (2EO/5EO) or derivatives thereof. More preferably said mixture is a mixture of 50% 2EO and 50% 5EO and most preferably a corresponding mixture of Oleyl/Cetylalcoholethoxylate.

In another embodiment the culture medium of the present invention comprises at least two fatty components. Said fatty components comprise but are not limited to those described herein supra. As referred to in this context, the two or more fatty components can be contained within one single molecule, for example, in form of a chemical structure comprising several distinct side residues or side chains or within a mixture of various components each of them comprising one or more of said fatty components. In a preferred embodiment a first fatty component comprises a fatty acid and a second fatty component comprises an emulsifying agent or derivative thereof.

In another embodiment the present invention relates to the culture medium as described herein, wherein the fatty component is present in an amount of 0.02-2.0% w/v, preferably 0.1 to 1.0%, more preferably 0.2 to 0.5% and most preferred the fatty component is present in an amount of 0.25% (w/v).

In a further embodiment the culture medium of the present invention further comprises:

| | | |
|---|---|---|
| peptone | 0.5-1.0% | w/v |
| yeast extract | 0.5-1.0% | w/v |
| D-glucose | 0.1% | w/v |
| trace element solution | 0.01% | v/v |
| K₂HPO₄ | 0.1-0.2% | w/v |

In another embodiment the trace element solution comprises the components listed in the following table:

| | | |
|---|---|---|
| Na₂-EDTA | 5.2 | g |
| FeSO₄ × 7H₂O | 2.1 | g |
| H₃BO₃ | 30.0 | mg |
| MnCl₂ × 4H₂O | 100.0 | mg |
| CoCl₂ × 6H₂O | 190.0 | mg |
| NiCl₂ × 6H₂O | 24.0 | mg |
| CuCl₂ × 2H₂O | 2.0 | mg |
| ZnSO₄ × 7H₂O | 144.0 | mg |
| Na₂MoO₄ × 2H₂O | 36.0 | mg |
| H₂O | add. 1000 | ml |

In a preferred embodiment the pH of said trace element solution is 6.5.

In another embodiment the culture medium of the present invention has a final pH of about 7.5 to 8.5, in a preferred embodiment of 8.5.

In the course of performing the method of the present invention it also turned out that the presence of Na⁺, NO₃⁻, Mg²⁺ in the form of NaNO₃, MgCl₂ and MgSO₄, respectively is advantageous for the growth characteristics of the microorganisms to be detected. Accordingly, the culture medium of the present invention preferably contains one or more of NaNO₃, MgCl₂ or MgSO₄. More preferably, the culture medium comprises NaNO₃ in a concentration of 0.05-0.1% w/v, MgCl₂ in a concentration of 0.01-0.05% w/v and/or MgSO₄×7H₂O in a concentration of 0.01-0.1% w/v.

The culture medium used in accordance with the present invention may be both, a specific culture medium for bacteria and specific culture medium for the culture of fungi. Both culture media provide for the same basic composition, but vary in the supplements used to distinguish between the colonizing microorganism, which can be bacteria or fungi. For example, a bacteria specific culture medium in accordance with the present invention may comprise fungicide and a redox colour, wherein a fungi specific culture medium may comprise anti-microbial agents such as antibiotics. Selective agents, and in particular anti-microbial and anti-fungal agents referred to herein also as antibiotics and fungicides, which inhibit or prevent growth of non-target organisms may also be comprised by the culture medium of the present invention. The decision as to which anti-microbial agent might be preferred should be based on the individual needs and experience. For all media types, optimal concentrations of anti-microbial and anti-fungal agents should be determined empirically. Many selective agents may be provided, and selective agents used in accordance with the present invention depend upon the targeted microorganism.

Preferably the selective agents include but are not limited to one or more of the following in concentrations within the following ranges: amikacin sulfate (about 0.0045 to 0.0055 g/l), the fungicide amphotericin B (about 0.00198 to 0.00242 g/l), and bacitracine (about 0.000476 to 0.00794 g/l), gentamicin Sulfate (5-50 μg/ml), kanamycin sulfate (100 μg/ml), nystatin (100 U/ml), penicillin G (50-100 U/ml), polymixin B sulfate (100 U/ml), streptomycin sulfate (50-100 μg/ml) and neomycin sulphate 50 μg/ml. Alternatively, thallium acetate, cycloheximide, tetracyclin, colistin, ansiomycin or clindamycin may be substituted. In particular, the anti-microbial agent comprises streptomycin. Preferably, streptomycin is used in a concentration of 0.003% w/v. In another preferred embodiment the anti-fungal agent comprises cycloheximid.

Preferably, the culture medium of the invention further comprises a redox indicator. The redox indicator in accordance with the present invention is contained in the medium in an amount which is sufficient to display growth of the target microorganism by exhibiting detectable characteristic signals produced in the medium during growth. The redox colour alters a detectable characteristic of the sample when the culture medium is metabolized by the target microorganisms. Therefore, it may be used to confirm the presence or absence of the target microorganisms in a colonized material. In particular, the redox indicator is triphenyltetrazolium chloride (TTC) which may be contained in the culture medium of the present invention in amount of preferably about 0.005 to 0.01% (w/v).

Hence, in one embodiment of the present invention the culture medium is a chromogenic culture medium for the simple and fast detection of microorganisms using chromogenic substrates. The chromogenic mixture contains chromogenic substrates as Salmon-GAL, X-Gal, X-glucuronide etc. Certain enzymes produced by, for example, some bacteria cleave the substrate, resulting in the different colouration of certain bacteria colonies; see for example Fluka, Buchs S G, Switzerland and Riedel-de Haën-Sigma-Aldrich Laborchemikalien GmbH, Seelze, Germany.

In another embodiment the culture medium of the invention is a solid culture medium which comprises agar. Preferably, the agar content is 1.0-1.5% w/v. The use of agar containing culture media in accordance of the present invention comprises pouring of the liquefied culture medium into suitable culture dishes (culture plates), wherein the material to be testified is applied onto the hardened surface of the cooled agar plates. Alternatively, the liquid culture medium can be poured onto a defined amount of sample to be tested with subsequent mixing of the sample and the culture medium in the agar plates pursuant to the Koch's postulate.

In another embodiment the culture medium of the present invention is dried. Said dry media include but are not limited to powders, reconstituable lyophilisates and the like.

Evaluation of the microbial count can be performed utilizing various means and methods which are dependent or independent of laboratory equipment using supporting devices. Thus, in one embodiment the present invention relates to a device comprising a culture medium or one or more of the components of the present invention. Some devices may have a generally flat horizontal surface which is divided into a plurality of recessed wells. Others have one or more surfaces with reagent island(s) immobilized thereon. Each well or reagent island is adapted to hold an aliquot of liquid. The wells or reagent islands are sized and shaped, and formed of a suitable material, to hold the aliquot within the well or reagent island by surface tension. Said device can be, for example, a dip slide, culture flask, culture dish (comprising pour-plate method according to the Koch's postulate and smear method) or detection stick.

In a particular preferred embodiment of the present invention, said device is a dip slide. Dip slides are well known to the person skilled in the art and usually consist of a plastic carrier coated with a sterile culture medium, which is dipped into the material to be tested, which is preferably in liquid form. The technical built-up of said dip slides does not differ from those of others commercially available dip slides which comprise a transparent plastic test tube having an external screw thread on the top for closing the top cover and a plastic device, the dip slide itself, which is fixed to the inside of the top cover. After applying of the sample the dip slide is then incubated to allow microbial growth and the resulting colonies are estimated, for example, by reference to a chart on which the density of the resulting colonies is compared to a reference chart to indicate the level of bacterial contamination. Results are expressed in terms of colony-forming units (CFU) per millilitre of fluid.

Such dip slides make determination of microbial counts fast and easy as they can be provided complete and ready to use. No special equipment or training is necessary. For review of dip slide technique see for example homepage of Filtertechnik, Hydrotechnik UK Limited, London, UK.

In a particular preferred embodiment of the dip slide of the present invention, both measurements for bacteria and fungi can be made simultaneously. In this embodiment, each side of the paddle is coated with a different medium of the present invention, one side being selective for fungi and the other for bacteria. Almost all aerobic bacteria will grow on the side designated for those organisms. Their growth can be in the form of red dots (colonies) on the surface of the agar. The paddle is compared to a chart in order to get a quantitative interpretation of the results. On the side designated for fungi, growth of yeast and mold will generally appear as cottony, filamentous structures. The reacted paddle is compared to a color chart from which an estimated fungal count can be made.

Generally, dip slides of the present invention will have a shelf life of one month, preferably three months and most preferably of at least six months. They can be stored in the refrigerator or at room temperature, but care should be taken to avoid frequent temperature changes.

In another embodiment of the present invention, the detection device is in the form of strips such as plastic strips to which nutrient-containing filter paper is attached. These strips are also complete and ready to use without any special equipment required.

Further methods and assay devices for the detection of the presence or amount of microorganism(s) in a sample, which can be adapted for the purposes of the present invention are described in the prior art; see. e. g., international application WO99/21655 which inter alia describes devices having a generally flat horizontal surface which is divided into a plurality of recessed wells.

In addition, the devices according to the present invention can be configured as described in U.S. Pat. No. 5,770,393 which describes a bacteria impermeable container and ampule, respectively, containing the liquid growth medium and a substrate-indicator complex. The complex includes a substrate component, e.g., starch, and an indicator molecule, e.g., a dye, a fluorescent molecule, or the like, which are tightly bound and complexed, but which are cleavable by a preselected enzyme. A sterilant passes over a carrier for microorganisms which, upon germination, are capable of rapidly generating large quantities of the preselected enzyme. Following the sterilization process, the carrier is immersed in the liquid growth medium. Any viable surviving microorganisms grow, generating the preselected enzyme. The enzymes cleave the bound indicator molecule from the substrate, resulting in a measurable property change in a couple of hours. Typical property changes include fluorescence, a color change, a change in pH which triggers a pH indicator color change, and the like. In an embodiment of the present invention the liquid medium contained in said container or ampule is a medium as described hereinbefore.

In another embodiment the present invention relates to a kit comprising the culture medium of the present invention or one or more components thereof or the device of the present invention. In a preferred embodiment the kit of the present invention comprises at least one fatty component and the anti-microbial or anti-fungal agent, each of them referred to supra. In a particular preferred embodiment the kit of the present invention further comprises means for the detection or determination of microorganisms. Such means and methods for detection or determination of microorganisms comprise manual or computer supported automated measurement of colony forming units (CFUs), photometrical turbidimetry and the like. In a most preferred embodiment the kit of the present invention further comprises a reference sample for the detection or determination of microorganisms which may be used as described herein above.

The kit of the present invention may further contain reagents and/or detection means, for example incubators, culture tubes such as sulfite reducing culture tubes, standard coolant, reference charts, refractometer 0-15%, hardness, pH, pipettes, etc. These common reagents and devices are commercially available; see for example Biosan Laboratories, Inc., MI, USA; Tektrak, UK, which also offer appropriate fungicides and biocides.

In an other embodiment the present invention is directed to a method of detecting contamination of metalworking fluids with microorganisms, said method comprising subjecting a sample of the metalworking fluid to the culture medium or the device of the present invention under conditions suitable for microorganisms to grow. In a preferred embodiment the method of the present invention is the microbial count indicative for the degree of contamination. Such methods include but are not limited to the steps of liquefying the sample (if necessary) and distributing the liquefied sample over the surface of the assay device or in the culture flask. Any excess liquid from the liquefied sample is drained from the surface of the device. The method then involves incubating the assay device until the presence or amount of the biological material, analyte, or microorganism is determined.

For example, microbial comparison charts for bacteria, yeast (single cell fungi) and mold, each in CFU per ml, can be easily prepared beforehand and evaluated; see for example the Microbial Comparison Chart provided by Troy Chemie GmbH, Germany. Interpretation of the results is industry, use, and environment specific. Metalworking fluids are a prime example. In this respect, bacterial counts of less than 10⁵ are typically not a major concern, as long as the counts are controlled with periodic antimicrobial dosing. But the same level in packaged products, where microbes may nourish and multiply for months unchecked, can literally destroy adhesives, paints and coatings. In these cases, a count of 10³ or above will generally require preservative treatment. A count of 10⁵ or greater will almost always require treatment.

The two most common tests for microbial monitoring include plate counts and dip slide tests. Plate counts involve growing a culture using a sample of the fluid. Microorganism colonies that grow on the plate are later counted and identified; see, e.g., Bienkowski “Coolants & Lubricants—Staying Pure” Manufacturing Engineering (1993), 55-61. Like plate counts, dip slide tests also involve growing cultures using a sample of the fluid. Dip slides provide a more simple, rapid screening method since cultures are grown overnight and a visual approximation is used to assess microbial contamination. When rancidity is a problem, microbial-growth dip slide monitoring provides a chance to add biocide before problems arise.

Weekly or biweekly monitoring is typically recommended for detection of microbial contamination, especially during the early stages of developing a fluid management program.

Once contamination in the fluid such as a coolant has been determined, the fluid may be cleaned, exchanged or biocides may be added according to methods well known in the art. For example, U.S. Pat. No. 6,126,843 describes a process for treating cooling lubricants to prevent attack by microorganisms using crospovidone-iodine as biocidal substance, wherein the cooling lubricant to be treated is brought into contact with a filter cake which consists essentially of particulate crospovidone-iodine as filter medium. A method of preventing microbial growth in oil-water metalworking fluid in a machine which comprises adding a partitionable anti-microbial agent to the lubricating oil or hydraulic or tapping fluid is described in US patent application US2005/059559. Another method generally used for treating contaminated cooling lubricants is to pass them through filters consisting of kieselguhr/perlite or other filtration aids, for example pure, crosslinked polyvinylpyrrolidone (PVPP). German patent application DE 196 200 84 describes the treatment of cooling lubricants in depth-type filters, wherein the depth-type filters contain a biocidal substance, for example crospovidone-iodine, which is embedded as particles in a framework of fibrous materials such as cellulose fibers.

Hence, in a further aspect the present invention relates to a process for treating metalworking fluids such as cooling lubricants to prevent attack by microorganisms using a biocidal substance, wherein the biocide addition to the aqueous process liquid, e.g. circulating washing water in paint plant or cooling lubricant involves determining the presence and/or amount of a microorganism in accordance with a method of the present invention as described hereinabove. One advantage of determining contamination of the fluid with microorganisms prior to the addition of a biocide is that dependent on the level of contamination and type of microorganism the biocide can be selectively chosen and applied in an appropriate and effective amount.

Hence, the present invention relates to the use of the culture medium or one or more components thereof, the device or the kit of the present invention for the method of the present invention.

Controlling pollution caused by hydrocarbon compounds, for example crude oil pollution, in soils and waters by bioremediation is acquiring increasing significance. Major advantages are afforded by the inexpensive in situ processes where no space is required for waste disposal. Microorganisms which consume hydrocarbon compounds are valuable tools in the context of this technology, even under comparatively unfavorable basic conditions, providing their enrichment and/or their growth at the point of pollution can be sufficiently stimulated. The working principle of bioremediation is based on optimal promotion of the growth of the pollution-consuming microorganism populations; see for review, e.g., U.S. Pat. No. 5,635,392.

In view of the fact that the media of the present invention are particular suited for the growth of microorganisms which colonize coolants because of their capability of utilizing carbon compounds such as present in oil and the like, it may also be advantageously used as a nutrient, preferably in combination with microorganism concentrates capable of degrading hydrocarbon compounds, for bioremediation, for example elimination of oil-wetted cuttings from geological land-supported or offshore drilling, for example from the development of geological occurrences.

Hence, in another aspect, the present invention relates to use of the media of the present invention for stimulation and as a growth aid for the accelerated growth of hydrocarbon-consuming microorganisms for their use in the biological degradation of organic components. In one embodiment of this aspect of the present invention, the medium is used together with microorganism concentrates which have been obtained by separate cultivation of natural strains isolated from hydrocarbon-contaminated localities of natural origin. It may be preferable in this regard to use concentrates of corresponding microorganism strains which in turn form biosurfactants as metabolism products. Without any claim to completeness, some possible starter cultures are listed in the following, although they are generally not used as isolated strains, but rather in the form of a mixture of a number of strains: Pseudomonas oleovorans DSM 1045; Pseudomonas putida DSM 548 and DSM 50208; Acinetobacter calcoaceticus DSM 590; Nocardia paraffineus ACC 21198; Arthrobacter paraffineus ATCC 15591. Information on the particular technology of bioremediation to be used can be found in the relevant literature, see for example Bourquin, Biofuture (1990), 24-35 or U.S. Pat. No. 5,635,392.

Most of the embodiments of the present invention have been described with respect their use in relation to metalworking fluids. However, other working fluids may be assessed as well for example coolants, cooling lubricants, borehole flushing fluids, cutting fluids, rolling fluids, hydraulic fluids, heat transfer media and wood protection agents.

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

Several documents are cited throughout the text of this specification. The contents of all cited references (including literature references, issued patents, published patent applications as cited throughout this application and manufacturer's specifications, instructions, etc.) are hereby expressly incorporated by reference; however, there is no admission that any document cited is indeed prior art as to the present invention.

The above disclosure generally describes the present invention. A more complete understanding can be obtained by reference to the following specific examples which are provided herein for purposes of illustration only and are not intended to limit the scope of the invention.

### EXAMPLES

The examples which follow further illustrate the invention, but should not be construed to limit the scope of the invention in any way. Detailed descriptions of conventional methods, such as those employed herein can be found in the cited literature; see also “The Merck Manual of Diagnosis and Therapy” Seventeenth Ed. ed by Beers and Berkow (Merck & Co., Inc. 2003).

The practice of the present invention will employ, unless otherwise indicated, conventional techniques of microbiology, which are within the skill of the art. Methods in cultivating microorganisms are described generally in the current editions of Molecular Cloning: A Laboratory Manual, (Sambrook et al., (1989) Molecular Cloning: A Laboratory Manual, 2nd ed., Cold Spring Harbor Laboratory Press); Current Protocols in Molecular Biology and Short Protocols in Molecular Biology, 3rd Edition (Ausubel et al., eds.); Brock Biology of Microorganisms, 10th ed. Michael Madigan, John M. Martinko, Jack Parker, Prentice Hall, 2002; Microbiology: An Introduction, 7th ed. Gerard Tortora, Berdell R. Funcke, Christine Case. Pearson Benjamin Cummings, 2000; Foundations in Microbiology, 4th ed. Kathleen Park, Talaro, Arthur Talaro. McGraw Hill, 2002; Bailey and Scott's Diagnostic Microbiology, 10th ed. Betty A. Forbes, Daniel F. Sahm, Ernest Trevino, Alice S. Weissfeld. Mosley, 1998, and Fundamental Techniques in Cell Culture . . . a Laboratory Handbook by SIGMA, Sigma-Aldrich; see supra.

### Example 1

### Preparation of Agar Plates Containing a Culture Medium of the Invention

For preparation of the culture medium the components have been mixed with demineralised or distilled water in order of the list below.

| | | |
|---|---|---|
| 0.25% | (w/v) | fatty acid |
| 0.25% | (w/v) | emulsifier (mixture of cetyl- and oleyl alcohol |
| | | ethoxylate) |
| 0.5% | (w/v) | peptone from casein |
| 0.1% | (w/v) | D-glucose |
| 0.1% | (w/v) | yeast extract |
| 0.2% | (w/v) | K₂HPO₄ |
| 0.01% | (w/v) | trace element solution |
| 0.01% | (w/v) | triphenyl tetrazolium chloride (TTC) |
| 0.003% | (w/v) | streptomycin, optional, to prevent bacterial growth for |
| | | selective enrichment of fungi |
| 1.5% | (w/v) | agar |

In particular, the following medium may be prepared:

| | | |
|---|---|---|
| 0.25% | (w/v) | linoleic acid, eicosanoic acid or palmitic acid |
| 0.25% | (w/v) | emulsifier (1:1 mixture of cetyl- and oleyl alcohol |
| | | ethoxylate) |
| 0.5% | (w/v) | peptone from casein |
| 0.1% | (w/v) | D-glucose |
| 0.1% | (w/v) | yeast extract |
| 0.2% | (w/v) | K₂HPO₄ |
| 0.01% | (w/v) | trace element solution |
| 0.01% | (w/v) | triphenyl tetrazolium chloride (TTC) |
| 0.003% | (w/v) | streptomycin |
| 1.5% | (w/v) | agar |

The mixture is stirred vigorously on a magnetic stirrer to emulsify the fatty acids well. After addition of all components the pH was adjusted to 8.4-8.5 using 1 M NaOH solution.

The culture medium was autoclaved at 121° C. for 15 min for sterilisation purposes. After cooling down to approximately 40° C. the culture medium was poured into sterile petri dishes and stored at 4° C. following an additional cooling period.

For the total enumeration of aerobic microorganisms no selection agent may be added to the base culture medium. For the specific detection of Pseudomonas cetrimide may be added, while for the specific detection of moulds and yeasts streptomycin may be used.

The use of agar plates is described in several pertinent textbooks; see for example Sambrook et al., (1989); supra. Such solid media plates allow the precise enumeration of microorganisms in fluids such as coolants by simply incubating a sample for about a few hours up to 3 days. When grown on plates, the microorganisms are characterized by their typical morphology. Also, it is easy to isolate a single colony for further confirmation. The plates can also be specially designed to accommodate 47 mm diameter filter membranes.

For storage purposes plates, e.g. with a 55 mm diameter, can be presented in a blister pack and individually protected with a separate plastic bag. Avoiding fast dehydration and contamination enhances the shelf life of the media.

### Example 2

### Preparation of Agar Plates Containing a Culture Medium of the Invention, Particularly Suitable for Detecting Mycobacteria

For preparation of a culture medium of the invention which is particularly suitable for detecting mycobacteria in the contaminated metal working fluids, the following components have to be mixed with demineralised distilled water in order of the list below:

| | | |
|---|---|---|
| 0.2-2.0% | (w/v) | fatty acids (C14-C20) |
| 0.2-2.0% | (w/v) | fatty alcohol ethoxylate (50% 2EO/50% 5EO) |
| 0.5-1.0% | (w/v) | peptone |
| 0.5-1.0% | (w/v) | yeast extract |
| 0.05-1.0% | (w/v) | D-glucose |
| 0.2-0.5% | (w/v) | glycerol |
| 0.05-0.2% | (w/v) | K₂HPO₄ |
| 0.05-0.1% | (w/v) | NaNO₃ |
| 0.01-0.05% | (w/v) | MgCl₂ |
| 0.01-0.1% | (w/v) | MgSO₄ × 7 H₂O |
| 0.005-0.01% | (w/v) | TTC |
| 0.01% | (w/v) | trace element solution |
| 1.0-1.5% | (w/v) | agar |

pH 7.5-8.5

Before adding the heat-instable components the culture medium is autoclaved at 121° C. for 15 minutes for sterilisation purposes. Subsequently the sterile filtered, heat-instable components are added and after cooling down to approximately 40° C. the culture medium was poured into sterile petri dishes and stored at 4° C. following an additional cooling period.

Any further usage of the agar plates can be performed as defined in example 1.

### Example 3

### Detection of Microorganisms

For preparing a petri dish or dip slide nutritive agar gel is dipped into an aqueous sample or the sample is applied to it. Some of the microbes adhere to the dish and slide, respectively, and reproduce during incubation to yield visible spots which are colonies. The pattern of spots is compared to a calibration chart and the initial number of microbes is read off the chart.

It should be noted that if the calibration and dip slides have been designed for aqueous samples; the chart will give misleading information if the slide is dipped into or through fuel or lubricating oil. Sterile disposable 1 ml Pasteur pipettes can be used to access water below a fuel sample and apply it to a slide; the original calibration is then valid. Some nutritive agars are designed to grow bacteria, whilst others grow moulds and yeasts, but neither type do this exclusively. Some dip slides will have different types of agar on opposite sides of the slide.

With respect to tests for bacteria most dip slides for bacteria incorporate a dye which stains the bacterial colonies red. The slides are incubated in a warm room for 2-3 days and the result read without opening the container.

Typical numbers of colonies expected per ml sample are:

Potable water 0-10²
Clean sea water 10²-10³
Polluted water 10³-10⁴
Lightly infected fuel in water bottom 10⁵
Heavily infected fuel in water bottom 10⁶-10⁸

With respect to tests for moulds and yeasts agars designed for yeasts and moulds will grow the former as round colonies, coloured white, cream or red. After incubation for 3-5 days under warm room conditions, the result is read from the calibration chart. The number of yeast colonies is usually less numerous than those of bacteria.

Typical numbers of colonies expected per ml sample are:

Potable water 0
Clean sea water 0-10²
Polluted water 0-10³
Lightly infected yeasts in fuel water bottom 10³-10⁴
Heavily infected yeasts in fuel water bottom 10⁴-10⁶

Moulds cannot be quantified in the same way as bacteria and yeasts; a single mould may be in the sample as a minute spore (not significant) or a ‘mat’ of proliferating strands. Incubation for yeasts is similar. Colonies are seen as large ‘furry’ patches, usually cream, green or grey/brown in color. Should any yeasts or moulds be detected, evidence of growth mats in the sample should be looked for.

It should be noted that agars designed for yeasts and moulds may contain the dye Rose Bengal, which colors the colonies pink and masks their real color. Rose Bengal is affected by undue exposure to light, after which it tends to suppress colony development. Thus, a slide should be checked with a sample known to contain yeasts or moulds (real ale or stale milk can be tried).

1: A method of detecting contamination of metal working fluids with microorganisms, said method comprising subjecting a sample of the fluid to a culture medium under conditions suitable for microorganisms to grow, wherein said culture medium comprises at least one fatty acid and an emulsifying agent.

2: The method of claim 1, wherein the microbial count is indicative for the degree of contamination.

3: The method of claim 1, wherein said fatty acids in the culture medium are selected from the group consisting of all even-numbered fatty acids C14 to C20.

4: The method of claim 1, wherein the fatty acids in the culture medium are saturated, mono- or polyunsaturated.

5: The method of claim 1, wherein the emulsifying agent or derivative thereof in the culture medium comprises fatty alcohols.

6: The method of claim 1, wherein the emulsifying agent in the culture medium comprises glycerol or a derivative thereof.

7: The method of claim 1, wherein the emulsifying agent in the culture medium comprises fatty alcohol ethoxylate or derivatives thereof.

8: The method of claim 1, wherein the emulsifying agent in the culture medium comprises a mixture of 2 ethylene oxide/5 ethylene oxide fatty alcohol ethoxylate or derivatives thereof

9: The method of claim 1, wherein the fatty acids in the culture medium are present in an amount of 0.2-2.0% w/v.

10: The method of claim 1, wherein the culture medium further comprises: peptone 0.5-1.0% w/v D-glucose 0.1% w/v trace element solution 0.01% v/v K2HPO4 0.1-0.2% w/v

11: The method of claim 1, wherein the trace element solution in the culture medium comprises the components listed in the following table: Na2-EDTA 5.2 g FeSO4×7H2O 2.1 g H3B03 30.0 mg MnC12×4H2O 100.0 mg CoC12×6H2O 190.0 mg NiC12×6H2O 24.0 mg CuC12×2H2O 2.0 mg ZnSO4×7H2O 144.0 mg Na2MoO4×2H2O 36.0 mg H2O add. 1000 ml

12: The method of claim 11, wherein the pH of said trace element solution is 6.5.

13: The method of claim 1, wherein the culture medium has a final pH of 7.5 to 8.5.

14: The method of claim 1, wherein the culture medium further comprises an anti-microbial or anti-fungal agent.

15: The method of claim 14, wherein the anti-microbial agent comprises streptomycin or the anti-fungal agent comprises cycloheximid.

16: The method of claim 14, wherein the culture medium further comprises a redox indicator.

17: The method of claim 16, wherein the redox indicator is triphenyltetrazolium chloride (TTC).

18: The method of claim 1, wherein the culture medium further comprises 1.0-1.5% w/v agar.

19: The method of claim 1, wherein the culture medium is dried.

20: A culture medium as defined in claim 1.

21: Device comprising a culture medium as defined in claim 1 or one or more components thereof.

22: The device of claim 21, which is a dip slide, culture flask, culture dish or detection stick.

23: Kit comprising a culture medium as defined in claim 1.

24: The kit of claim 23 comprising at least one fatty acid and the anti-microbial or anti-fungal agent.

25: The kit of claim 23 further comprising means for the detection or determination of microorganisms comprising mycobacteria.

26: The kit of claim 23 further comprising a reference sample for the detection or determination of microorganisms.

27. (canceled)
